(19) United States
(12) Patent Application Publication
(10) Pub. No.: US 2015/0250785 A1
(43) Pub. Date: Sep. 10, 2015

(54) Tropomyosin-Related Kinase Inhibitors

(71) Applicant: PFIZER LIMITED, Kent, England (GB)

(72) Inventors: Mark David Andrews, Sandwich (GB); Sharanjeet Kaur Bagal, Great Abington (GB); Sarah Elizabeth Skerratt, Great Abington (GB)

(73) Assignee: Pfizer Limited, Sandwich, Kent, UK (GB)

(21) Appl. No.: 14/430,584

(22) PCT Filed: Sep. 26, 2013
(86) PCT No.: PCT/IB2013/058887

§ 371 Date: Mar. 24, 2015

Related U.S. Application Data
(60) Provisional application No. 61/709,513, filed on Oct. 4, 2012.

(51) Int. Cl. A61K 31/4985; A61K 31/437; C07D 487/04; C07D 471/04

(52) U.S. Cl. CPC A61K 31/4985; C07D 471/04; A61K 31/437; C07D 487/04

(57) ABSTRACT

The present invention relates to compounds of Formula (IA) and (IB)

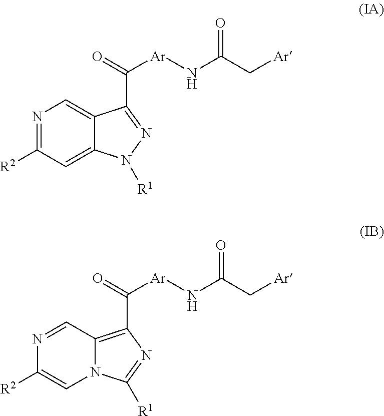

and their pharmaceutically acceptable salts, wherein the substituents are as described herein, and their use in medicine, in particular as Trk antagonists.

The invention described herein relates to certain heterocyclic compounds and the pharmaceutically acceptable salts of such compounds. The invention also relates to the processes for the preparation of the compounds, compositions containing the compounds, and the uses of such compounds and salts in treating diseases or conditions associated with tropomyosin-related kinase (Trk), activity. More specifically the invention relates to the compounds and their salts useful as inhibitors of Trk.

### BACKGROUND

Tropomyosin-related kinases (Trks) are a family of receptor tyrosine kinases activated by neurotrophins. Trks play important roles in pain sensation as well as tumour cell growth and survival signaling. Thus, inhibitors of Trk receptor kinases might provide targeted treatments for conditions such as pain and cancer. Recent developments in this field have been reviewed by Wang et al in Expert Opin. Ther. Patents (2009) 19(3): 305-319 and an extract is reproduced below.

### “1.1 Trk Receptors

As one of the largest family of proteins encoded by the human genome, protein kinases are the central regulators of signal transduction as well as control of various complex cell processes. Receptor tyrosine kinases (RTKs) are a subfamily of protein kinases (up to 100 members) bound to the cell membrane that specifically act on the tyrosine residues of proteins. One small group within this subfamily is the Trk kinases, with three highly homologous isoforms: TrkA, TrkB, and TrkC. All three isoforms are activated by high affinity growth factors named neurotrophins (NT): i) nerve growth factor (NGF), which activates TrkA; ii) brain-derived neurotrophic factor (BDNF) and NT-4/5, which activate TrkB; and iii) NT-3, which activates TrkC. The binding of neurotrophins to the extracellular domain of Trks causes the Trk kinase to autophosphorylate at several intracellular tyrosine sites and triggers downstream signal transduction pathways. Trks and neurotrophins are well known for their effects on neuronal growth and survival.

### 1.2 Trks and Cancer

Originally isolated from neuronal tissues, Trks were thought to mainly affect the maintenance and survival of neuronal cells. However, in the past 20 years, increasing evidence has suggested that Trks play key roles in malignant transformation, chemotaxis, metastasis, and survival signaling in human tumors. The association between Trks and cancer focused on prostate cancer in earlier years and the topic has been reviewed. For example, it was reported that malignant prostate epithelial cells secrete a series of neurotrophins and at least one Trks. In pancreatic cancer, it was proposed that paracrine and/or autocrine neurotrophin-Trk interactions may influence the invasive behavior of the cancer. TrkB was also reported to be overexpressed in metastatic human pancreatic cancer cells. Recently, there have been a number of new findings in other cancer settings. For example, a translocation leads to expression of a fusion protein derived from the N-terminus of the ETV6 transcription factor and the C-terminal kinase domain of TrkC. The resulting ETV6-TrkC fusions are oncogenic in vitro and appear causative in secretory breast carcinoma and some acute myelogenous leukemias (AML). Constitutively active TrkA fusions occurred in a subset of papillary thyroid cancers and colon carcinomas. In neuroblastoma, TrkB expression was reported to be a strong predictor of aggressive tumor growth and poor prognosis, and TrkB overexpression was also associated with increased resistance to chemotherapy in neuroblastoma tumor cells in vitro. One report showed that a novel splice variant of TrkA called TrkAIII signaled in the absence of neurotrophins through the inositol phosphate-AKT pathway in a subset of neuroblastoma. Also, mutational analysis of the tyrosine kinome revealed that Trk mutations occurred in colorectal and lung cancers. In summary, Trks have been linked to a variety of human cancers, and discovering a Trk inhibitor and testing it clinically might provide further insight to the biological and medical hypothesis of treating cancer with targeted therapies.

### 1.3 Trks and Pain

Besides the newly developed association with cancer, Trks are also being recognized as an important mediator of pain sensation. Congenital insensitivity to pain with anhidrosis (CIPA) is a disorder of the peripheral nerves (and normally innervated sweat glands) that prevents the patient from either being able to adequately perceive painful stimuli or to sweat. TrkA defects have been shown to cause CIPA in various ethnic groups.

Currently, non-steroidal anti-inflammatory drugs (NSAIDs) and opiates have low efficacy and/or side effects (e.g., gastrointestinal/renal and psychotropic side effects, respectively) against neuropathic pain and therefore development of novel pain treatments is highly desired. It has been recognized that NGF levels are elevated in response to chronic pain, injury and inflammation and the administration of exogenous NGF increases pain hypersensitivity. In addition, inhibition of NGF function with either anti-NGF antibodies or non-selective small molecule Trk inhibitors has been shown to have effects on pain in animal models. It appears that a selective Trk inhibitor (inhibiting at least NGF's target, the TrkA receptor) might provide clinical benefit for the treatment of pain. Excellent earlier reviews have covered targeting NGF/BDNF for the treatment of pain so this review will only focus on small molecule Trk kinase inhibitors claimed against cancer and pain. However, it is notable that the NGF antibody tanezumab was very recently reported to show good efficacy in a Phase II trial against osteoarthritic knee pain.”

International Patent Application publication number WO2009/012283 refers to various fluorophenyl compounds as Trk inhibitors; International Patent Application publication numbers WO2009/152087, WO2008/080015 and WO2008/08001 and WO2009/152083 refer to various fused pyrroles as kinase modulators; International Patent Application publication numbers WO2009/143024 and WO2009/143018 refer to various pyrrolo[2,3-d]pyrimidines substituted as Trk inhibitors; International Patent Application publication numbers WO2004/056830 and WO2005/116035 describe various 4-amino-pyrrolo[2,3-d]pyrimidines as Trk inhibitors. International Patent Application publication number WO2011/133637 describes various pyrrolo[2,3-d]pyrimidines and pyrrolo[2,3-b]pyridines as inhibitors of various kinases. International Patent Application publication number WO2005/099709 describes bicyclic heterocycles as serine protease inhibitors. International Patent Application publication number WO2007/047207 describes bicyclic heterocycles as FLAP modulators.

US provisional application U.S. 61/471,758 was filed 5 Apr. 2011. Convention applications U.S. Ser. No. 13/439,131 (filed 4 Apr. 2012) and PCT/IB2012/051363 (filed 22 Mar. 2012) claiming priority thereto. The whole contents of those application in their entirety are herewith included by reference thereto.

Thus Trk inhibitors have a wide variety of potential medical uses. There is a need to provide new Trk inhibitors that are good drug candidates. In particular, compounds should preferably bind potently to the Trk receptors in a selective manner compared to other receptors, whilst showing little affinity for other receptors, including other kinase and/or GPC receptors, and show functional activity as Trk receptor antagonists. They should be non-toxic and demonstrate few side-effects. Furthermore, the ideal drug candidate will exist in a physical form that is stable, non-hygroscopic and easily formulated. They should preferably be e.g. well absorbed from the gastrointestinal tract, and/or be injectable directly into the bloodstream, muscle, or subcutaneously, and/or be metabolically stable and possess favourable pharmacokinetic properties.

Among the aims of this invention are to provide orally-active, efficacious, compounds and salts which can be used as active drug substances, particularly Trk antagonists, i.e. that block the intracellular kinase activity of the Trk, e.g. TrkA (NGF) receptor. Other desirable features include good HLM/hepatocyte stability, oral bioavailability, metabolic stability, absorption, selectivity over other types of kinase, dofetilide selectivity. Preferable compounds and salts will show a lack of CYP inhibition/induction, and be CNS-sparing.

### SUMMARY

The present invention provides compounds of Formula IA and IB

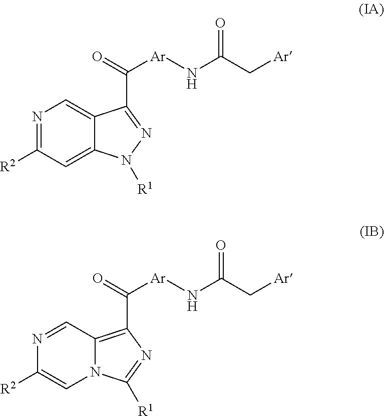

or a pharmaceutically acceptable salt thereof, wherein
R¹ is C₂₋₄ alkyl optionally substituted by 1 or 2 OH, optionally wherein a methylene group is replaced by an oxetane group,
or R¹ is C₄₋₆ cycloalkyl optionally substituted by OH;

### R² is H or NH₂;

Ar is a ring system selected from

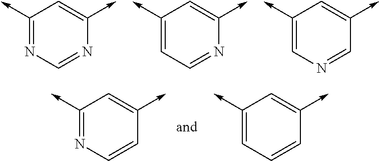

which ring system is optionally substituted on a carbon atom by CN, C₁₋₃ alkyl, C₁₋₃ alkoxy, or C₃₋₆ cycloalkyloxy;
and Ar′ is a ring system selected from

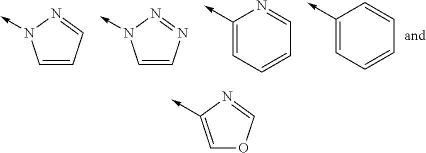

which ring system is optionally substituted on a carbon atom by 1 or 2 substituents independently selected from:
halo, ═O, CN, C₁₋₃ alkyl optionally substituted by one or more F. C₁₋₃ alkoxy optionally substituted by one or more F, C₃₋₆ cycloalkyl, C₃₋₆ cycloalkyloxy and SO₂(C₁₋₃ alkyl).

Compounds of formula I herein can refer to compounds of formula IA and/or of formula IB.

The invention also comprises pharmaceutical compositions comprising a therapeutically effective amount of a compound of formula I as defined herein, or a pharmaceutically acceptable salt thereof, and a pharmaceutically acceptable carrier.

The invention is also directed to a method of treating a disease or condition indicated for treatment with a Trk antagonist, in a subject, by administering to a subject in need thereof a therapeutically effective amount of one or more of the compounds herein, or a pharmaceutically acceptable salt thereof.

Other aspects of the invention will be apparent from the remaining description and claims.

Preferably, the compounds of the present invention are potent antagonists at Trk receptors, and have a suitable PK profile to enable once daily dosing.

The compounds of the present invention are potentially useful in the treatment of a range of disorders where a Trk antagonist is indicated, particularly pain indications. Depending on the disease and condition of the patient, the term “treatment” as used herein may include one or more of curative, palliative and prophylactic treatment.

According to the invention a compound of the present invention may be useful to treat any physiological pain such as inflammatory pain, nociceptive pain, neuropathic pain, acute pain, chronic pain, musculo-skeletal pain, on-going pain, central pain, heart and vascular pain, head pain, orofacial pain. Other pain conditions which may be treated include intense acute pain and chronic pain conditions which may involve the same pain pathways driven by pathophysiological processes and as such cease to provide a protective mechanism and instead contribute to debilitating symptoms associated with a wide range of disease states.

Pain is a feature of many trauma and disease states. When a substantial injury, via disease or trauma, to body tissue occurs the characteristics of nociceptor activation are altered, this leads to hypersensitivity at the site of damage and in nearby normal tissue. In acute pain the sensitivity returns to normal once the injury has healed. However, in many chronic pain states, the hypersensitivity far outlasts the healing process and is normally due to nervous system injury due to maladaptation of the afferent fibres (Woolf & Salter 2000 Science 288: 1765-1768). Clinical pain is present when discomfort and abnormal sensitivity feature among the patient's symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Pain can be divided into a number of different areas because of differing pathophysiology, these include nociceptive, inflammatory, neuropathic pain among others. It should be noted that some types of pain have multiple aetiologies and thus can be classified in more than one area, e.g. Back pain, Cancer pain have both nociceptive and neuropathic components.

### Nociceptive Pain

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmit rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumbar facet joints, sacroiliac joints, paraspinal muscles or the posterior longitudinal ligament.

### Neuropathic Pain

According to the invention a compound of the present invention can potentially be used to treat neuropathic pain and the symptoms of neuropathic pain including hyperalgesia, allodynia and ongoing pain. Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

### Intense Acute Pain and Chronic Pain

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) exaggerated pain responses to noxious stimuli (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies.

### Chronic Pain

Chronic pain comprises one or more of, chronic nociceptive pain, chronic neuropathic pain, chronic inflammatory pain, breakthrough pain, persistent pain hyperalgesia, allodynia, central sensitisation, peripheral sensitisation, disinhibition and augmented facilitation.

Chronic pain includes cancer pain, e.g. cancer pain arising from malignancy, adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, anemia, bladder cancer, bone cancer, brain cancer, breast cancer, carcinoid tumour s, cervical cancer, chemotherapy, colon cancer, cytopenia, endometrial cancer, esophageal cancer, gastric cancer, head cancer, neck cancer, hepatobiliary cancer, kidney cancer, leukemia, liver cancer, lung cancer, lymphoma, Hodgkin's disease, lymphoma, non-Hodgkin's, nervous system tumours, oral cancer, ovarian cancer, pancreatic cancer, prostate cancer, rectal cancer, skin cancer, stomach cancer, testicular cancer, thyroid cancer, urethral cancer, bone cancer, sarcomas cancer of the connective tissue, cancer of bone tissue, cancer of blood-forming cells, cancer of bone marrow, multiple myeloma, leukaemia, primary or secondary bone cancer, tumours that metastasize to the bone, tumours infiltrating the nerve and hollow viscus, tumours near neural structures. Cancer pain also comprises visceral pain, e.g. visceral pain which arises from pancreatic cancer and/or metastases in the abdomen, somatic pain, e.g. somatic pain due to one or more of bone cancer, metastasis in the bone, postsurgical pain, sarcomas cancer of the connective tissue, cancer of bone tissue, cancer of blood-forming cells of the bone marrow, multiple myeloma, leukaemia, primary or secondary bone cancer.

### Inflammatory Pain

Inflammatory conditions include acute inflammation, persistent acute inflammation, chronic inflammation, and combined acute and chronic inflammation.

Inflammatory pain includes acute inflammatory pain and/or chronic inflammatory pain wherein the chronic inflammatory pain can be pain involving both peripheral and central sensitisation and/or mixed etiology pain involving both inflammatory pain and neuropathic pain or nociceptive pain components. Inflammatory pain also comprises hyperalgesia, e.g. primary and/or secondary hyperalgesia. Additionally or alternatively the inflammatory pain can include allodynia. Inflammatory pain also comprises pain that persists beyond resolution of an underlying disorder or inflammatory condition or healing of an injury.

Inflammatory pain is pain resulting an inflammatory condition. e.g. in response to acute tissue injury due to trauma, disease e.g. an inflammatory disease, immune reaction, the presence of foreign substances, chemicals or infective particles for example micro-organisms. Inflammatory conditions can be either acute or chronic inflammation or both.

Inflammatory pain can result from an inflammatory condition due to an inflammatory disease such as inflammatory joint diseases, inflammatory connective tissue diseases, inflammatory autoimmune diseases, inflammatory myopathies, inflammatory digestive system diseases, inflammatory air way diseases, cellular immune inflammation diseases, hypersensitivities and allergies, vasular inflammation diseases, non-immune inflammatory disease, synovitis, villonodular synovitis, arthralgias, ankylosing spondylitis, spondyloarthritis, spondyloarthropathy, gout, Pagets disease, periarticular disorders such as bursitis, rheumatoid disease, rheumatoid arthritis and osteoarthritis, rheumatoid arthritis or osteoarthritis. Rheumatoid arthritis in particular, represents ongoing inflammation associated with severe pain. Arthritic pain is a form of inflammatory pain and arises from inflammation in a joint which causes both peripheral sensitization and central sensitization. Under inflammatory conditions the nociceptive system is activated by normally innocuous and nonpainful mechanical stimuli.

Additionally when the joint is at rest pain is present and appears as spontaneous pain and hyperalgesia (augmented pain response on noxious stimulation and pain on normally nonpainful stimulation). Inflammatory processes in peripheral tissues lead to central sensitization in the spinal cord, which contributes to hyperalgesia and allodynia typically associated with inflammatory pain. Other types of inflammatory pain include inflammatory bowel diseases (IBD).

### Other Types of Pain

Other types of pain include but are not limited to:
Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis;
Central pain or ‘thalamic pain’ as defined by pain caused by lesion or dysfunction of the nervous system including but not limited to central post-stroke pain, multiple sclerosis, spinal cord injury, Parkinson's disease and epilepsy;
Heart and vascular pain including but not limited to angina, myocardical infarction, mitral stenosis, pericarditis, Raynaud's phenomenon, scleredoma, scleredoma, skeletal muscle ischemia;
Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis;

Head pain including but not limited to migraine, migraine with aura, migraine without aura cluster headache, tension-type headache. Orofacial pain including but not limited to dental pain, temporomandibular myofascial pain, tinnitus, hot flushes, restless leg syndrome and blocking development of abuse potential. Further pain conditions may include, back pain (e.g. chronic lower back pain), cancer pain, complex regional syndrome, HIV-related neuropathic pain, post-operative induced neuropathic pain, post-stroke pain, spinal cord injury pain, traumatic nerve injury pain, diabetic peripheral neuropathy, moderate/severe interstitial cystitis pain, irritable bowel syndrome pain, moderate/severe endometriosis pain, moderate/severe pelvic pain, moderate/severe prostatitis pain, moderate/severe osteoarthritis pain, post-herpetic neuralgia, rheumatoid arthritis pain, dysmenorrhea pain, pre-emptive post-operative pain, trigeminal neuralgia, bursitis, dental pain, fibromyalgia or myofacial pain, menstrual pain, migraine, neuropathic pain (including painful diabetic neuropathy), pain associated with post-herpetic neuralgia, post-operative pain, referred pain, trigeminal neuralgia, visceral pain (including interstitial cystitis and IBS) and pain associated with AIDS, allodynia, burns, cancer, hyperalgesia, hypersensitisation, spinal trauma and/or degeneration and stroke.

### DETAILED DESCRIPTION

Embodiment 1 of the invention is a compound of Formula IA or IB:

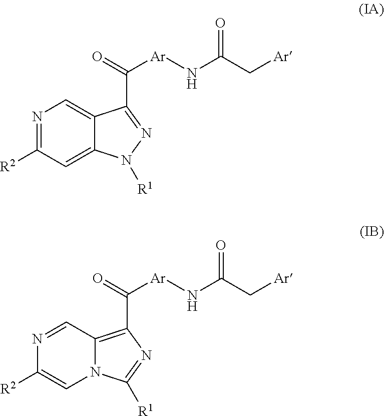

or a pharmaceutically acceptable salt thereof, wherein
R¹ is C₂₋₄ alkyl optionally substituted by 1 or 2 OH, optionally wherein a methylene group is replaced by an oxetane group,
or R¹ is C₄₋₆ cycloalkyl optionally substituted by OH;

### R² is H or NH₂;

Ar is a ring system selected from

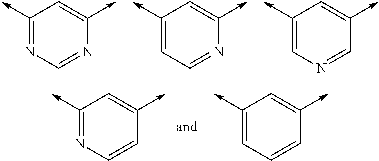

which ring system is optionally substituted on a carbon atom by CN, C₁₋₃ alkyl, C₁₋₃ alkoxy, or C₃₋₆ cycloalkyloxy;
and Ar′ is a ring system selected from

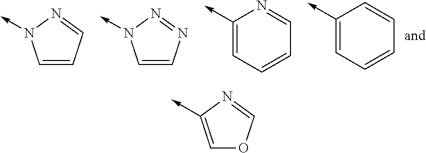

which ring system is optionally substituted on a carbon atom by 1 or 2 substituents independently selected from:
halo, ═O, CN, C₁₋₃ alkyl optionally substituted by one or more F, C₁₋₃ alkoxy optionally substituted by one or more F, C₃₋₆ cycloalkyl, C₃₋₆ cycloalkyloxy and SO₂(C₁₋₃ alkyl).

Embodiment 2 is a compound or salt according to embodiment 1 wherein R¹ is selected from:

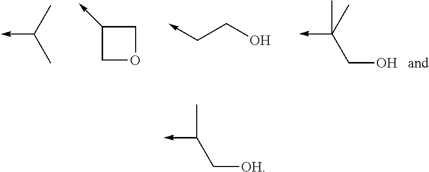

Embodiment 3 is a compound or salt according to embodiment 1 or 2 wherein Ar′ is a ring system which ring is selected from:

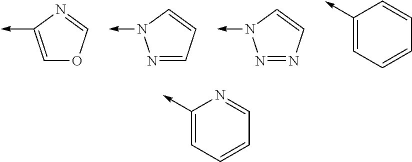

and which ring is optionally substituted by 1 or 2 substituents independently selected from F, Cl, ═O, CN, CF₃, OCF₃, CH₃, isopropyl, OCH₃, cyclopropyl, and

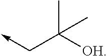

Embodiment 4 is a compound or salt according to embodiment 1, 2 or 3 wherein Ar is

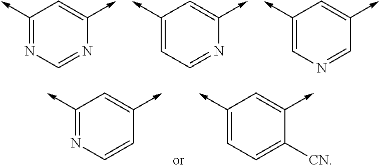

Embodiment 5 is a compound or salt according to embodiment 1, 2, 3 or 4 wherein R¹ is 1-hydroxy-2-methylpropan-2-yl, 1-hydroxypropan-2-yl or isopropyl.

Embodiment 6 is a compound or salt according to embodiment 1, 2, 3, 4 or 5 wherein R² is H.

Embodiment 7 is a compound selected from:
2-(2-cyclopropyl-1,3-oxazol-4-yl)-N-{4-[(1-isopropyl-1H-pyrazolo[4,3-c]pyridin-3-yl)carbonyl]pyridin-2-yl}acetamide;
2-(4-cyanophenyl)-N-{4-[(1-isopropyl-1H-pyrazolo[4,3-c]pyridin-3-yl)carbonyl]pyridin-2-yl}acetamide; and
N-{4-[(3-isopropylimidazo[1,5-a]pyrazin-1-yl)carbonyl]pyridin-2-yl}-2-[3-(trifluoromethyl)-1H-pyrazol-1-yl]acetamide,
   or a pharmaceutically acceptable salt thereof.

Embodiment 8 is a pharmaceutical composition comprising a compound of the formula (IA or IB) or a pharmaceutically acceptable salt thereof, as defined in any one of the preceding embodiments 1 to 7, and a pharmaceutically acceptable carrier.

Embodiment 9 is a compound of the formula (IA or IB) or a pharmaceutically acceptable salt thereof, as defined in any one of embodiments 1 to 7, for use as a medicament.

Embodiment 10 is a compound of formula (IA or IB) or a pharmaceutically acceptable salt thereof, as defined in any one of embodiments 1 to 7 for use in the treatment of a disease for which an Trk receptor antagonist is indicated.

1. Embodiment 11 is a compound of formula (IA or IB) or a pharmaceutically acceptable salt thereof, as defined in any one of embodiments 1 to 7 for use in the treatment of pain or cancer.

Embodiment 12 is the use of a compound of the formula (IA or IB) or a pharmaceutically acceptable salt or composition thereof, as defined in any one of embodiments 1 to 7, for the manufacture of a medicament to treat a disease for which an Trk receptor antagonist is indicated.

Embodiment 13 is the use of a compound of the formula (IA or IB) or a pharmaceutically acceptable salt or composition thereof, as defined in any one of embodiments 1 to 7, for the manufacture of a medicament to treat pain or cancer.

Embodiment 14 is a method of treatment of a mammal, to treat a disease for which an Trk receptor antagonist is indicated, comprising treating said mammal with an effective amount of a compound of the formula (IA or IB) or a pharmaceutically acceptable salt thereof, as defined in any one of embodiments 1 to 7.

Embodiment 15 is a method of treatment of pain or cancer in a mammal, comprising treating said mammal with an effective amount of a compound of the formula (IA or IB) or a pharmaceutically acceptable salt thereof, as defined in any one of embodiments 1 to 7.

Embodiment 16 is a compound or salt according to any one of embodiments 1 to 7 for use in a medical treatment in combination with a further drug substance.

Further embodiments include:

A compound or salt according to any of embodiments 1 to 6 where R¹ is isopropyl;

A compound or salt according to any of embodiments 1 to 5 where R² is NH₂;

A compound or salt according to any one of embodiments 1 to 6 wherein Ar is

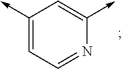

A compound or salt according to any one of embodiments 1 to 6 wherein Ar′ is (2-cyclopropyl-1,3-oxazol-4-yl), (4-cyanophenyl), or (3-(trifluoromethyl)-1H-pyrazol-1-yl);

Any novel genus of intermediates described in the Schemes below;

Any novel specific intermediate described in the Preparations below;

Any novel process described herein.

“Halogen” means a fluoro, chloro, bromo or iodo group.

“Alkyl” groups, containing the requisite number of carbon atoms, can be unbranched or branched. Examples of alkyl include methyl, ethyl, n-propyl, i-propyl, n-butyl, i-butyl, sec-butyl and t-butyl.

“Pharmaceutically acceptable salts” of the compounds of formula I include the acid addition and base addition salts (including disalts, hemisalts, etc.) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base addition salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

The compounds of the invention include compounds of formula I and salts thereof as hereinbefore defined, polymorphs, and isomers thereof (including optical, geometric and tautomeric isomers) as hereinafter defined and isotopically-labelled compounds of formula I.

Unless otherwise specified, compounds of formula (I) containing one or more asymmetric carbon atoms can exist as two or more stereoisomers. Where a compound of formula (I) contains for example, a keto or guanidine group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of formula (I), including compounds exhibiting more than one type of isomerism, and mixtures of one or more thereof. Also included are acid addition or base addition salts wherein the counterion is optically active, for example, D-lactate or L-lysine, or racemic, for example, DL-tartrate or DL-arginine.

Examples of types of potential tautomerisms shown by the compounds of the invention include hydroxypyridine [symbol] pyridone; amide [symbol] hydroxyl-imine and keto [symbol] enol tautomersims:

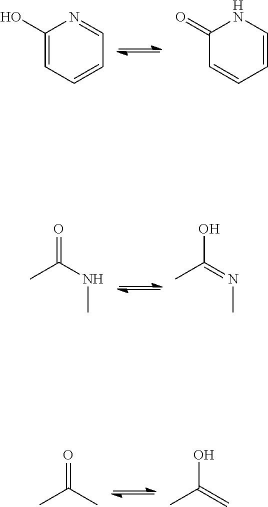

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or other derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagents in place of the non-labelled reagent previously employed.

The routes below, including those mentioned in the Examples and Preparations, illustrate methods of synthesising compounds of formula (I). The skilled person will appreciate that the compounds of the invention, and intermediates thereto, could be made by methods other than those specifically described herein, for example by adaptation of the methods described herein, for example by methods known in the art. Suitable guides to synthesis, functional group interconversions, use of protecting groups, etc., are for example: “Comprehensive Organic Transformations” by R C Larock, VCH Publishers Inc. (1989); Advanced Organic Chemistry” by J. March, Wiley Interscience (1985); “Designing Organic Synthesis” by S Warren, Wiley Interscience (1978); “Organic Synthesis—The Disconnection Approach” by S Warren, Wiley Interscience (1982); “Guidebook to Organic Synthesis” by R K Mackie and D M Smith, Longman (1982); “Protective Groups in Organic Synthesis” by T W Greene and PGM Wuts, John Wiley and Sons, Inc. (1999); and “Protecting Groups” by P J, Kocienski, Georg Thieme Verlag (1994); and any updated versions of said standard works.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

In the general synthetic methods below, unless otherwise specified, the substituents are as defined above with reference to the compounds of formula (I) above.

Where ratios of solvents are given, the ratios are by volume.

The compounds of the invention may be prepared by any method known in the art for the preparation of compounds of analogous structure. In particular, the compounds of the invention can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of formula (I). It will be further appreciated that it may be necessary or desirable to carry out the transformations in a different order from that described in the schemes, or to modify one or more of the transformations, to provide the desired compound of the invention.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

Where ratios of solvents are given, the ratios are by volume.

According to a first process, compounds of formula (IA) may be prepared by the process illustrated in Scheme 1.

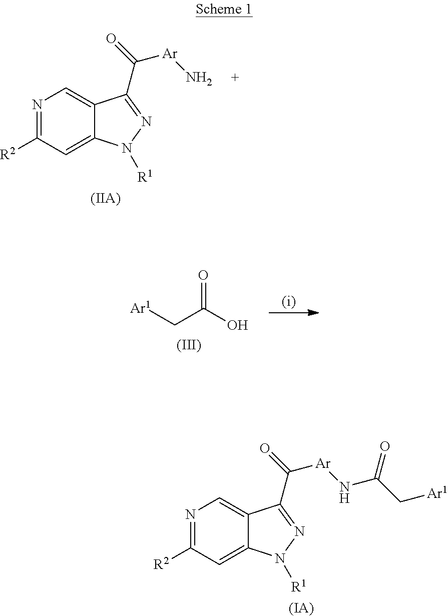

Compounds of formula (IA) may be prepared from compounds of formula (IIA) according to process step (i), an amide bond formation step, if necessary adding a suitable base (such as DIPEA) and/or additive (such as 4-dimethylaminopyridine).

Typical conditions employed involve stirring the amine of general formula (IIA) and the acid of general formula (III) together with a suitable coupling reagent such as HATU or HBTU or 1-propylphosphonic acid cyclic anhydride, if necessary adding a suitable base such as NMM, DIPEA or TEA in a suitable solvent such as pyridine, THF or DMA at a temperature from room temperature up to 70° C. A suitable alternative is to use an additive (such as 4-dimethylaminopyridine) as well as a base. Any suitable solvent may be used in place of those mentioned above. At least one equivalent of the acid (III) and at least one equivalent of the coupling reagent should be used and an excess of one or both may be used if desired.

Where R¹ contains a suitable hydroxyl protecting group in intermediate (IIA), removal of the protecting group (PG) can be done in situ or as an additional step, adding a suitable acid and organic solvent to the crude residue after the amide bond formation has taken place. Common protecting groups to use include TBDMS or TMS, which are readily removed by treatment with an acid such as aqueous hydrogen chloride or hydrogen chloride in an organic solvent such as THF or dioxane or by treatment with a fluoride source such as tetrabutylammonium fluoride in an organic solvent such as THF, and THP and dimethylacetal.

Intermediates of general formula (III) are either commercially available or will be well-known to those skilled in the art with reference to literature precedents and/or the preparations herein.

Compounds of general formula (IIA) are described in Schemes 2 and 3.

According to a second process, compounds of formula (IIA) may be prepared by the process illustrated in Scheme 2.

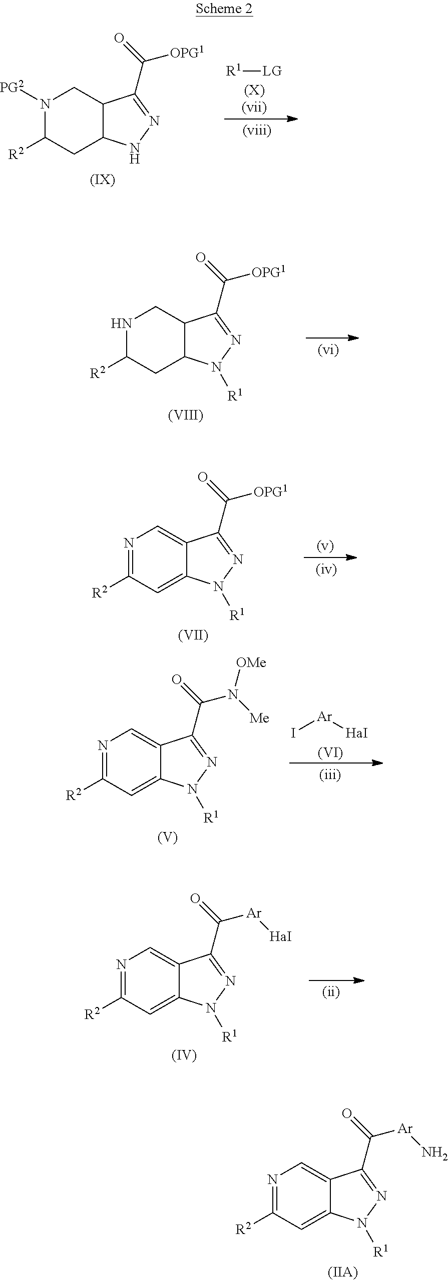

Wherein R² is H; Hal is Br or Cl, PG¹ is a suitable ester protecting group such as ethyl; and PG² is a suitable amino protecting group such as tert-butylcarbonate; LG is Cl, Br, I, or mesylate, tosylate or triflate;

Compounds of formula (IIA) may be prepared from compounds of formula (IV) according to process step (ii), a direct amination of the halide using standard literature conditions. For example, amine (IIA) is typically prepared using ammonia with a suitable copper catalyst such as copper (II) sulphate or copper (I) oxide in suitable solvent such as NMP in a sealed vessel at a temperature between room temperature and 140° C.

Compounds of formula (IV) may be prepared from compounds of formula (V) and (VI) according to process step (iii), a metallation of intermediate halide (VI) (using a suitable organometallic reagent such as butyllithium or isopropylmagnesium chloride) and reacting with the Weinreb amide intermediate (V) at a temperature from −78° C. up to room temperature in a suitable solvent such as THF or toluene. Preferred conditions comprise iPrMgCl in THF at −20° C.

Compounds of formula (VI) are commercially available.

Compounds of formula (V) may be prepared from compounds of formula (VII) according to process steps (v) and (iv), a base mediated hydrolysis step followed by an amide bond formation step.

Preferred conditions comprise sodium hydroxide in THF at reflux followed by HBTU with triethylamine and N-methoxy-N-methylamine hydrochloride in DCM at room temperature.

Compounds of formula (VII) may be prepared from compounds of formula (VIII) according to reaction step (vi), an oxidative aromatisation reaction in the presence of a hydrogenation catalyst, such as platinum, palladium, or nickel, and a suitable hydrogen acceptor such as maleic acid, cyclohexene or benzene at elevated temperatures. Typical conditions comprise 10% Pd/C in 4-isopropylbenzene at reflux.

Compounds of formula (VIII) may be prepared from compounds of formula (IX) and (X) according to reaction steps (vii) and (viii), an alkylation reaction in the presence of an inorganic base followed by an acid mediated deprotection reaction. Typical conditions comprise potassium carbonate in acetone at reflux with compounds of formula (X) followed by 4M HCl in dioxane at room temperature.

Compounds of formula (X) are commercially available.

Compounds of formula (IX) are well-known to those skilled in the art with reference to literature precedents and/or the preparations described herein from the cyclisation of hydrazine, diethyloxalate and tert-butyl 4-oxopiperidine-1-carboxylate.

According to a third process, compounds of formula (IIA) may be prepared by the process illustrated in Scheme 3.

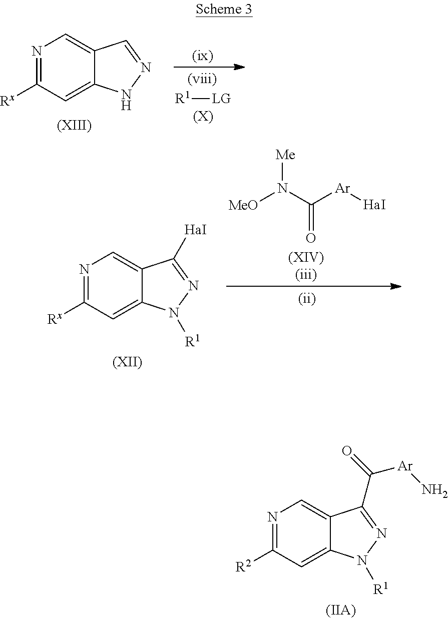

Wherein R^{x} is H or HaI, R² is H or NH₂; Hal is Cl, Br or I; LG is Cl, Br, I, or mesylate, tosylate or triflate;

Compounds of formula (IIA) may be prepared from compounds of formula (XII) and (XIV) according to process steps (iii) and (ii), a metallation of intermediate halide (XII) and reaction with Weinreb amide intermediate (XIV) as described in Scheme 2 step (iii), followed by a direct amination of the halide as described in Scheme 2 step (ii). Wherein R^{x} is HaI, conversion to R² as NH₂ may be achieved under the same amination conditions, or alternatively, two equivalents of 4-methoxybenzylamine or benzophenone imine may be used followed by a suitable acid mediated deprotection step.

Compounds of formula (XII) may be prepared from compounds of formula (XIII) and (X) according to process steps (ix) and (viii), an electrophilic halogenation reaction followed by an alkylation step in the presence of an inorganic base. Typical conditions comprise NIS or NBS in acetonitrile at room temperature followed by potassium carbonate with compounds of formula (X) in DMF at room temperature.

Compounds of formulae (X), (XIII) and (XIV) are either commercially available or well-known to those skilled in the art with reference to literature precedents.

According to a fourth process, compounds of formula (IB) may be prepared by the process illustrated in Scheme 4.

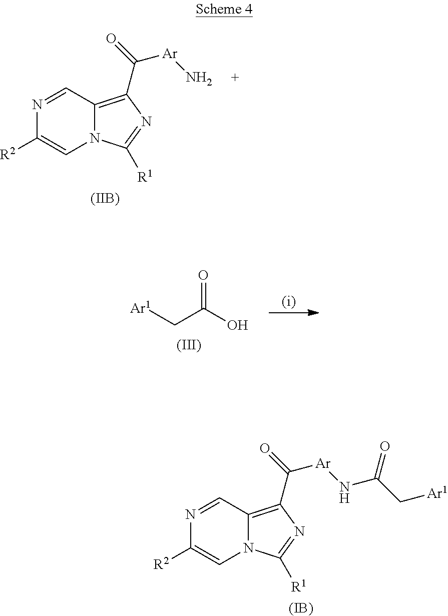

Compounds of formula (IB) may be prepared from compounds of formula (IIB) according to process step (i), an amide bond formation step as described in Scheme 1.

Where R¹ contains a suitable hydroxyl protecting group in intermediate (IIB), removal of the protecting group (PG) can be done in situ or as an additional step, adding a suitable acid and organic solvent to the crude residue after the amide bond formation has taken place. Common protecting groups to use include TBDMS or TMS, which are readily removed by treatment with an acid such as aqueous hydrogen chloride or hydrogen chloride in an organic solvent such as THF or dioxane or by treatment with a fluoride source such as tetrabutylammonium fluoride in an organic solvent such as THF, and THP and dimethylacetal.

According to a fifth process, compounds of formula (IIB) may be prepared by the process illustrated in Scheme 5.

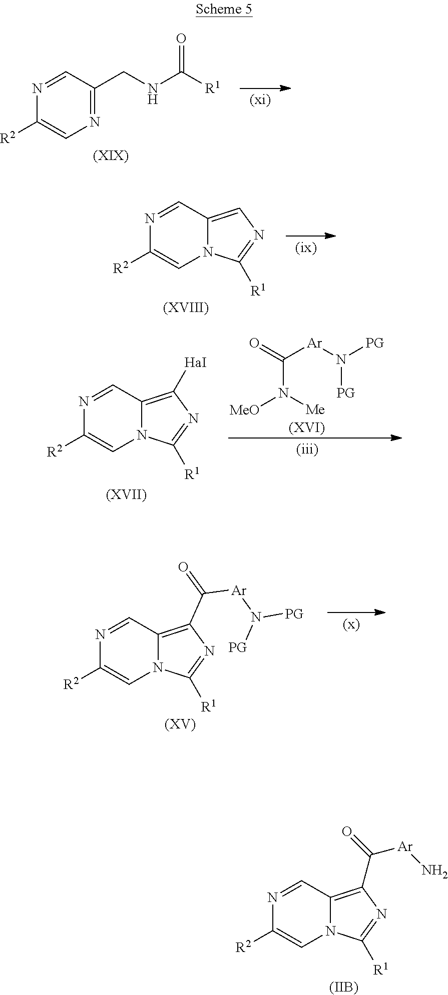

Wherein R² is H, PG is diphenylmethylene and Hal is Br or I;

Compounds of formula (IIB) may be prepared from compounds of formula (XV) according to process step (x), a deprotection step conveniently mediated under acidic conditions using acids such as HCl, TFA or citric acid. Wherein PG is diphenylmethylene, preferred conditions comprise a 1M aqueous solution of citric acid in THF at room temperature.

Compounds of formula (XV) may be prepared from compounds of formula (XVII) and (XVI) according to process step (iii), a metallation of intermediate halide (XVII) and reaction with Weinreb amide intermediate (XVI) as described in Scheme 2 step (iii). Preferred conditions comprise n-butyl lithium in anhydrous toluene at −78° C.

Compounds of formula (XVII) may be prepared from compounds of formula (XVIII) according to process step (ix), an electrophilic halogenation reaction as described in Scheme 3 step (ix). Preferred conditions comprise NIS in DMF at 60° C.

Compounds of formula (XVIII) may be prepared from compounds of formula (XIX) according to process step (xi), a cyclisation reaction in the presence of a dehydrating reagent such as POCl₃. Typical conditions comprise POCl₃ with catalytic DMF at 55° C.

Compounds of formula (XIX) and (XIV) are either commercially available or will be well-known to those skilled in the art with reference to literature precedents and/or the preparations herein.

According to a sixth process, compounds of formula (IIB) may be prepared by the process illustrated in Scheme 6.

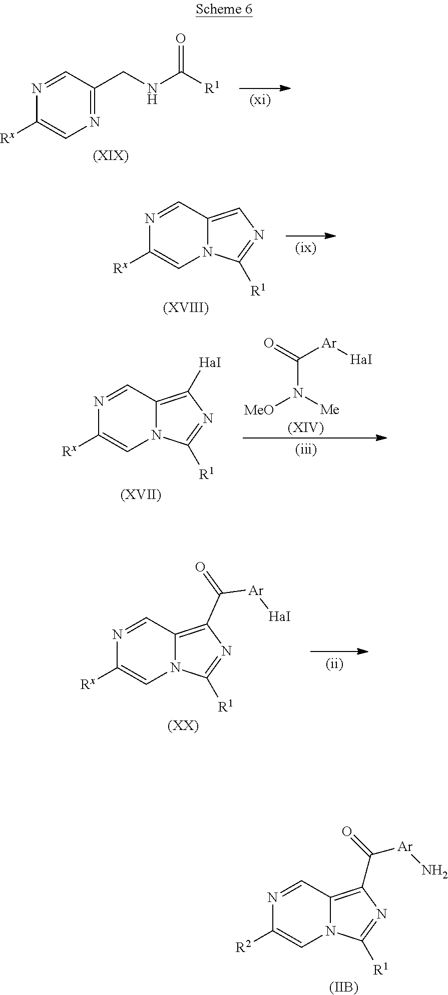

Wherein R² is H or NH₂, R^{x} is H or HaI, Hal is Cl, Br or I;

Compounds of formula (IIB) may be prepared from compounds of formula (XX) according to process step (ii) a direct amination of the halide (XX) as described in Scheme 3 step (ii). Wherein R^{x} is HaI, conversion to R² as NH₂ may be achieved under the same amination conditions. Alternatively two equivalents of 4-methoxybenzylamine or benzophenone imine may be used followed by a suitable acid mediated deprotection step.

Compounds of formula (XX) may be prepared from compounds of formula (XVII) and (XIV) according to process step (iii) a metallation of intermediate halide (XVII) and reaction with Weinreb amide intermediate (XIV) as described in Scheme 2 step (iii).

Compounds of formula (XVII) may be prepared from compounds of formula (XIX) as described in Scheme 5.

Compounds of formula (XIV) are either commercially available or well-known to those skilled in the art with reference to literature precedents.

According to a further embodiment the present invention provides novel intermediate compounds.

Pharmaceutically acceptable salts of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

The compounds of the invention intended for pharmaceutical use may be administered alone or in combination with one or more other compounds of the invention or in combination with one or more other drug agent (or as any combination thereof). Generally, they will be administered as a formulation in association with one or more pharmaceutically acceptable excipients. The term “excipient” is used herein to describe any biologically inactive ingredient other than the compounds and salts of the invention. The choice of excipient will to a large extent depend on factors such as the particular mode of administration, the effect of the excipient on solubility and stability, and the nature of the dosage form. For example, a compound of the formula I, or a pharmaceutically acceptable salt or solvate thereof, as defined above, may be administered simultaneously (e.g. as a fixed dose combination), sequentially or separately in combination with one or more other drug agent.

Exemplary additional agents could be selected from one or more of:
a Nav1.7 channel modulator, such as a compound disclosed in WO 2009/012242 or WO2010/079443;
an alternative sodium channel modulator, such as a Nav1.3 modulator (e.g. as disclosed in WO2008/118758); or a Nav1.8 modulator (e.g. as disclosed in WO 2008/135826, more particularly N-[6-Amino-5-(2-chloro-5-methoxyphenyl)pyridin-2-yl]-1-methyl-1H-pyrazole-5-carboxamide);
an inhibitor of nerve growth factor signaling, such as: an agent that binds to NGF and inhibits NGF biological activity and/or downstream pathway(s) mediated by NGF signaling (e.g. tanezumab), a TrkA antagonist or a p75 antagoinsist;
a compound which increases the levels of endocannabinoid, such as a compound with fatty acid amid hydrolase inhibitory (FAAH) activity, in particular those disclosed in WO 2008/047229 (e.g. N-pyridazin-3-yl-4-(3-{[5-(trifluoromethyl)pyridine-2-yl]oxy}benzylidene)piperidene-1-carboxamide);
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a 5-HT₃ antagonist, such as ondansetron
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-Nyrimidin-2-yl methyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxyl)pyridin-3-yl]carbonyl}amino)ethyl]benzoic acid;
a microsomal prostaglandin E synthase type 1 (mPGES-1) inhibitor;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
   a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504).

Pharmaceutical compositions suitable for the delivery of compounds and salts of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Compounds and salts of the invention intended for pharmaceutical use may be prepared and administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavoring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. [Make sure these specific ranges are relevant]

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

### Parenteral Administration

The compounds and salts of the invention may be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) and salts used in the preparation of parenteral solutions may be increased by the use of appropriate formulation techniques, such as the incorporation of solubility-enhancing agents.

Formulations for parenteral administration may be formulated to be immediate and/or modified release. Thus, compounds and salts of the invention may be formulated as a solid, semi-solid, or thixotropic liquid for administration as an implanted depot providing modified release of the active compound. An example of such formulations include drug-coated stents.

### Topical Administration

The compounds and salts of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated [see, for example, Finnin and Morgan, J Pharm Sci, 88 (10), 955-958 (October 1999).] Other means of topical administration include delivery by electroporation, iontophoresis, phonophoresis, sonophoresis and microneedle or needle-free (e.g. Powderject™, Bioject™, etc.) injection.

### Inhaled/Intranasal Administration

The compounds and salts of the invention may also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

A pressurised container, pump, spray, atomizer, or nebuliser may contain a solution or suspension of the compound(s) or salt(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound or salt of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound or salt of the invention per actuation and the actuation volume may vary from 1 μl to 100 μl. A typical formulation may comprise a compound of formula (I) or salt thereof, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by a prefilled capsule, blister or pocket or by a system that utilises a gravimetrically fed dosing chamber. Units in accordance with the invention are typically arranged to administer a metered dose or “puff” containing from 1 to 5000 μg of the compound or salt. The overall daily dose will typically be in the range 1 μg to 20 mg which may be administered in a single dose or, more usually, as divided doses throughout the day.

### Rectal/Intravaginal Administration

The compounds and salts of the invention may be administered rectally or vaginally, for example, in the form of a suppository, pessary, or enema. Cocoa butter is a traditional suppository base, but various well known alternatives may be used as appropriate.

### Ocular and Aural Administration

The compounds and salts of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid; a cellulosic polymer, for example, hydroxypropylmethylcellulose, hydroxyethylcellulose, or methyl cellulose; or a heteropolysaccharide polymer, for example, gelan gum, may be incorporated together with a preservative, such as benzalkonium chloride. Such formulations may also be delivered by iontophoresis.

### Other Technologies

The compounds and salts of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds and salts of the invention is typically in the range 0.1 mg to 200 mg depending, of course, on the mode of administration, preferred in the range 1 mg to 100 mg and more preferred in the range 1 mg to 50 mg. The total daily dose may be administered in single or divided doses.

These dosages are based on an average human subject having a weight of about 65 kg to 70 kg. The physician will readily be able to determine doses for subjects whose weight falls outside this range, such as infants and the elderly.

For the above-mentioned therapeutic uses, the dosage administered will, of course, vary with the compound or salt employed, the mode of administration, the treatment desired and the disorder indicated. The total daily dosage of the compound of formula (I)/salt/solvate (active ingredient) will, generally, be in the range from 1 mg to 1 gram, preferably 1 mg to 250 mg, more preferably 10 mg to 100 mg. The total daily dose may be administered in single or divided doses. The present invention also encompasses sustained release compositions.

The pharmaceutical composition may, for example, be in a form suitable for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

For parenteral dosages, this may conveniently be prepared as a solution or as a dry powder requiring dissolution by a pharmacist, medical practitioner or the patient. It may be provided in a bottle or sterile syringe. For example it may be provided as a powder in a multicompartment syringe which allows the dry powder and solvent to be mixed just prior to administration (to aid long-term stability and storage). Syringes could be used which allow multiple doses to be administered from a single device.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems.

Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, (1978). Pharmaceutical compositions are preferably manufactured under GMP conditions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system.

Compositions for sustained release or implantation may comprise pharmaceutically acceptable polymeric or hydrophobic materials such as an emulsion, an ion exchange resin, a sparingly soluble polymer, or a sparingly soluble salt.

The precise dosage administered of each active ingredient will vary depending upon any number of factors, including but not limited to, the type of animal and type of disease state being treated, the age of the animal, and the route(s) of administration.

The following non-limiting Preparations and Examples illustrate the preparation of compounds and salts of the present invention.

### GENERAL EXPERIMENTAL

Where singleton compounds have been analysed by LCMS, there are several methods used. These are illustrated below.

The invention is illustrated by the following non-limiting Examples in which the following abbreviations and definitions are used:

AcOH—acetic acid; APCI—atmospheric pressure chemical ionization; Arbocel is a filter agent; br s—broad singlet; BINAP—2,2′-bis(diphenylphosphino)-1,1′-binapthyl; nBuLi—n-Butyllithium; CDCl₃—deuterated chloroform; Cs₂CO₃ is caesium carbonate; CuI is copper (I) iodide; Cu(OAc)₂ is copper (II) acetate; δ—chemical shift; d—doublet; DAD—diode array detector; DCE—1,2-dichloroethane DCM—dichloromethane; DEA—diethylamine; DIBAL—Diisobutylaluminium hydride; DIPEA—diisopropylethylamine; DMAP—4-dimethylaminopyridine; DME—dimethoxyethane; DMF—N,N-dimethylformamide; DMF-DMA—N,N-dimethylformamide-dimethylacetal; DMSO—dimethylsulphoxide DPPF—1,1′-bis(diphenylphosphino)ferrocene; ELSD—evaporative light scattering detector; ESI—electrospray ionization; Et₂O—diethylether; EtOAc/EA—ethyl acetate; EtOH—ethanol; g—gram; HATU—2-(7-azabenzotriazol-1-yl)-1,1,3,3-tetramethyluronium hexafluorophosphate; HBTU is O-benzotriazol-1-yl-N,N,N′,N′-tetramethyluronium hexafluorophosphate; HCl is hydrochloric acid; HOBT is N-hydroxybenzotriazole hydrate; HPLC—high pressure liquid chromatography; IPA—isopropyl alcohol; K₂CO₃ is potassium carbonate; KHSO₄ is potassium hydrogen sulphate; KOAc is potassium acetate; KOH is potassium hydroxide; K₃PO₄ is potassium phosphate tribasic; KF—potassium fluoride; L is litre; LCMS—liquid chromatography mass spectrometry; LiHMDS—Lithium hexamethyldisilazide; m—multiplet; mg—milligram; mL—millilitre; M/Z—Mass Spectrum Peak; MeCN—acetonitrile; MeOH—methanol; 2-MeTHF—2-methyltetrahydrofuran; MgSO₄ is magnesium sulphate; MnO₂—manganese dioxide; NaClO₂—sodium chlorite; NaH—sodium hydride; NaHCO₃—sodium hydrogencarbonate; Na₂CO₃—sodium carbonate; NaH₂PO₄—sodium phosphate; NaHSO₃—sodium bisulphite; NaHSO₄—sodium hydrogensulphate; NaOH—sodium hydroxide; Na₂SO₄—sodium sulphate; NH₃—ammonia; NH₄Cl—ammonium chloride; NMM—N-MethylMorpholine; NMR—nuclear magnetic resonance; Pd/C—palladium on carbon; PdCl₂—palladium dichloride; Pd₂(dba)₃ is tris(dibenzylideneacetone)dipalladium(0); Pd(PPh₃)₄—palladium tetrakis(triphenylphosphine); Pd(OAc)₂—palladium acetate; PTSA—para-toluenesulfonic acid; Prep—preparation; Rₜ—retention time; q—quartet; s—singlet; TBDMS—tertbutyldimethylsilyl; TBME—tertbutyldimethylether; TCP—1-propylphosphonic acid cyclic anhydride; TEA—triethylamine; TFA—trifluoroacetic acid; THF—tetrahydrofuran; TLC—thin layer chromatography; (R,S)—racemic mixture; WSCDI—1-(3-dimethylaminopropyl)-3-ethylcarbodiimide hydrochloride.

For the avoidance of doubt, named compounds used herein have been named using IUAPC, Chemdraw and/or Name Pro ACD Labs Name Software v7.11™ or using other standard nomenclature. NMR spectra were measured in deuterated solvents and were consistent with the names/structures given below.

“CommAv” means a commercially available intermediate/reagent.

The Preparations and Examples that follow illustrate the invention but do not limit the invention in any way. All starting materials are available commercially or described in the literature. All temperature are in ° C. Flash column chromatography was carried out using Merck silica gel 60 (9385) or Redisep silica. NMR was carried out using a Varian Mercury 300/400 MHz NMR spectrometer or a Jeol ECX 400 MHz NMR.

The mass spectra were obtained using:

### Waters ZQ ESCI

### Applied Biosystem's API-2000 5 min LC-MS

Waters Alliance 2795 with ZQ2000 (ESI)

### Aglient 110 HPLC 5 min (System 5)

Where singleton compounds have been analysed by LCMS, there are four methods used. These are illustrated below:

### System 1

5 minute LC-MS gradient and instrument conditions
A: 0.05% formic acid in water
B: acetonitrile
Column: C18 phase XBridge 50×4.6 mm with 5 micron particle size
Gradient: 90-10% A over 3 min, 1 min hold, 1 min re-equilibration, 1.2 mL/min flow rate
UV: 200 nm-260 nm DAD

### Temperature: 25° C.

### System 2

5 minute LC-MS gradient and instrument conditions
A: 10 mM ammonium acetate in water
B: acetonitrile
Column: C18 phase Gemini NX 50×4.6 mm with 5 micron particle size
Gradient: 90-10% A over 3 min, 1 min hold, 1 min re-equilibration, 1.2 mL/min flow rate
UV: 200 nm-260 nm DAD

### Temperature: 25° C.

### System 3

5 minute LC-MS gradient and instrument conditions
A: 0.1% formic acid in water
B: 0.1% formic acid in acetonitrile
Column: C18 phase Waters Sunfire 50×4.6 mm with 5 micron particle size
Gradient: 95-5% A over 3 min, 1 min hold, 1 min re-equilibration, 1.5 mL/min flow rate
UV: 225 nm—ELSD-MS
Temperature: ambient

### System 4

5 minute LC-MS gradient and instrument conditions
A: 0.1% ammonium hydroxide in water
B: 0.1% ammonium hydroxide in acetonitrile
Column: C18 phase XTerra 50×4.6 mm with 5 micron particle size
Gradient: 95-5% A over 3 min, 1 min hold, 1 min re-equilibration, 1.5 mL/min flow rate
UV: 225 nm—ELSD-MS
Temperature: ambient

Where singleton compounds have been purified by High Performance Liquid Chromatography, unless otherwise stated, one of the following methods were used:

Waters Purification Systems with mass spec or UV detection

### Prep System 1

10 minute prep LC-MS gradient and instrument conditions
A: 0.1% formic acid in water
B: 0.1% formic acid in acetonitrile
Column: C18 phase Sunfire 100×19.0 mm or Gemini-NX 3 um C18 110A
Gradient: 95-2% A over 7 min, 2 min hold, 1 min re-equilibration, 18 mL/min flow rate
Temperature: ambient

### Prep System 2

10 minute prep LC-MS gradient and instrument conditions
A: 0.1% DEA in water
B: 0.1% DEA in acetonitrile
Column: C18 phase Xterra 100×19.0 mm or Gemini-NX 3 um C18 110A
Gradient: 95-2% A over 7 min, 2 min hold, 1 min re-equilibration, 18 mL/min flow rate
Temperature: ambient

### Example 1

### 2-(2-cyclopropyl-1,3-oxazol-4-yl)-N-{4-[(1-isopropyl-1H-pyrazolo[4,3-c]pyridin-3-yl)carbonyl]pyridin-2-yl}acetamide

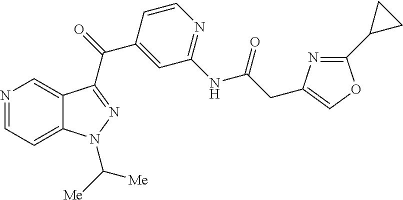 [SMILES: CC(C)n1nc(C(=O)c2ccnc(NC(=O)Cc3coc(C4CC4)n3)c2)c2cnccc21]

To a solution of (2-aminopyridin-4-yl)(1-isopropyl-1H-pyrazolo[4,3-c]pyridin-3-yl)methanone (Preparation 1, 23 mg, 0.082 mmol) in pyridine (1 mL) was added (2-cyclopropyl-1,3-oxazol-4-yl)acetic acid (Preparation 17, 13.7 mg, 0.082 mmol) and HATU (31.2 mg, 0.082 mmol) and the reaction was heated to 50° C. for 4 hours. Further equivalents of (2-cyclopropyl-1,3-oxazol-4-yl)acetic acid (13.7 mg, 0.082 mmol) and HATU (13.7 mg, 0.082 mmol) were added and the reaction heated to 50° C. for 7 hours followed by 4 days at room temperature. The reaction was partitioned between EtOAc and saturated aqueous NaHCO₃, the organic layer was collected, washed with brine, dried over MgSO₄ and concentrated in vacuo. The residue was purified using preparative HPLC to afford the title compound.

LCMS Rt=2.97 minutes MS m/z 431 [M+H]⁺

### Example 2

### 2-(4-cyanophenyl)-N-{4-[(1-isopropyl-1H-pyrazolo[4,3-c]pyridin-3-yl)carbonyl]pyridin-2-yl}acetamide

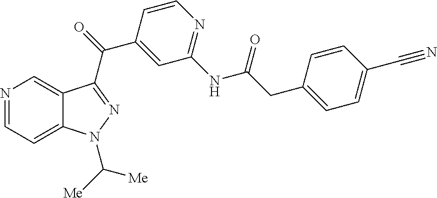 [SMILES: CC(C)n1nc(C(=O)c2ccnc(NC(=O)Cc3ccc(C#N)cc3)c2)c2cnccc21]

To a solution of (2-aminopyridin-4-yl)(1-isopropyl-1H-pyrazolo[4,3-c]pyridin-3-yl)methanone (Preparation 1, 23 mg, 0.082 mmol) in pyridine (1 mL) was added 4-cyanophenylacetic acid (14.5 mg, 0.090 mmol) and HATU (34.2 mg, 0.090 mmol) and the reaction was heated to 50° C. for 4 hours. Further equivalents of 4-cyanophenylacetic acid (13.7 mg, 0.082 mmol) and HATU (13.7 mg, 0.082 mmol) were added and the reaction heated to 50° C. for a further 3 hours before cooling to room temperature. The reaction was partitioned between EtOAc and saturated aqueous NaHCO₃, the organic layer was collected, washed with brine, dried over MgSO₄ and concentrated in vacuo. The residue was purified using preparative HPLC to afford the title compound.

LCMS Rt=2.49 minutes MS m/z 425 [M+H]⁺

### Example 3

### N-{4-[(3-isopropylimidazo[1,5-a]pyrazin-1-yl)carbonyl]pyridin-2-yl}-2-[3-(trifluoromethyl)-1H-pyrazol-1-yl]acetamide

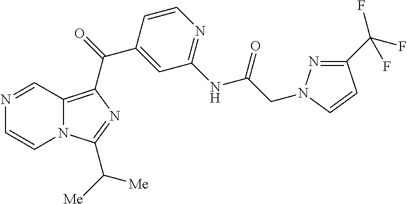 [SMILES: CC(C)c1nc(C(=O)c2ccnc(NC(=O)Cn3ccc(C(F)(F)F)n3)c2)c2cnccn12]

To a solution of (2-aminopyridin-4-yl)(3-isopropylimidazo[1,5-a]pyrazin-1-yl)methanone (Preparation 10, 80 mg, 0.283 mmol) in THF (2 mL) was added [4-(trifluoromethyl)-1H-pyrazol-1-yl)]acetic acid (Preparation 19, 55 mg, 0.283 mmol), 1-propylphosphonic acid cyclic anhydride (425 uL, 0.90 mmol) and triethylamine (138 ul, 0.99 mmol) and the reaction was heated to reflux for 48 hours. The reaction was cooled and concentrated in vacuo. The residue was partitioned between saturated aqueous NaHCO₃ solution and EtOAc. The organic layer was collected, washed with brine, dried over Na₂SO₄ and concentrated in vacuo. The residue was purified using silica gel column chromatography eluting with 60-65% EtOAc in hexanes followed by preparative HPLC to afford the title compound.

¹H NMR (400 MHz, DMSO-d₆): δ ppm 1.34 (s, 6H), 3.60 (m, 1H), 5.28 (s, 2H), 6.76 (s, 1H), 8.00 (m, 3H), 8.60 (m, 2H), 8.88 (br s, 1H), 9.61 (s, 1H), 11.14 (s, 1H).

LCMS Rt=3.09 minutes MS m/z 458 [M+H]⁺

### Preparation 1

### (2-aminopyridin-4-yl)(1-isopropyl-1H-pyrazolo[4,3-c]pyridin-3-yl)methanone

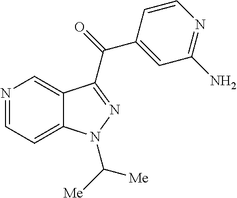 [SMILES: CC(C)n1nc(C(=O)c2ccnc(N)c2)c2cnccc21]

To a suspension of (2-bromopyridin-4-yl)(1-isopropyl-1H-pyrazolo[4,3-c]pyridin-3-yl)methanone (Preparation 2, 148 mg, 0.429 mmol) in 880 ammonia (5 mL) was added dioxane (enough to enable solubility) followed by copper sulphate (32 mg, 0.129 mmol) and the reaction was heated in a sealed vessel at 140° C. for 16 hours. The reaction was cooled, concentrated in vacuo and the residue stirred in 1N HCl for 30 minutes. Saturated aqueous NaHCO₃ was added until pH=7 and the mixture extracted with EtOAc three times (3×25 mL). The combined organic layers were washed with brine, dried over MgSO₄ and concentrated in vacuo to afford the title compound as a yellow oil (45 mg, 37%).

¹H NMR (400 MHz, CDCl₃): δ ppm 1.66 (s, 6H), 4.72 (br d, 2H), 4.96 (m, 1H), 7.37 (s, 1H), 7.42 (d, 1H), 7.56 (d, 1H), 8.27 (d, 1H), 8.57 (d, 1H), 9.75 (s, 1H).

MS m/z 282 [M+H]⁺

### Preparation 2

### (2-bromopyridin-4-yl)(1-isopropyl-1H-pyrazolo[4,3-c]pyridin-3-yl)methanone

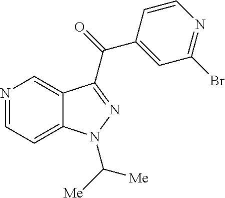 [SMILES: CC(C)n1nc(C(=O)c2ccnc(Br)c2)c2cnccc21]

To a solution of 2-bromo-4-iodopyridine (324 mg, 1.14 mmol) in THF (5 mL) at −20° C. was added ⁱPrMgCl.LiCl (221 mg, 1.52 mmol) and the reaction stirred for 30 minutes to reach −10° C. 1-isopropyl-N-methoxy-N-methyl-1H-pyrazolo[4,3-c]pyridine-3-carboxamide (Preparation 3, 189 mg, 0.761 mmol) was then added as a solution in THF (5 mL) and the reaction allowed to warm to room temperature for 18 hours. The reaction was quenched by the addition of saturated aqueous ammonium chloride solution and stirred for 10 minutes. The layers were separated and the aqueous layer extracted with EtOAc. The combined organic layers were washed with brine, dried over MgSO₄ and concentrated in vacuo. The residue was purified using silica gel column chromatography eluting with DCM:EtOAc 1:1 to afford the title compound as a yellow solid (263 mg, 55%).

¹H NMR (400 MHz, CDCl₃): δ ppm 1.72 (s, 6H), 4.98 (m, 1H), 7.46 (d, 1H), 8.15 (d, 1H), 8.42 (d, 1H), 8.56-8.63 (m, 2H), 9.76 (s, 1H).

MS m/z 345 [M⁷⁹Br+H]⁺, 347 [M⁸¹Br+H]⁺

### Preparation 3

### 1-isopropyl-N-methoxy-N-methyl-1H-pyrazolo[4,3-c]pyridine-3-carboxamide

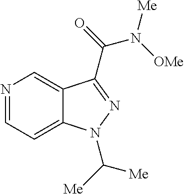 [SMILES: CON(C)C(=O)c1nn(C(C)C)c2ccncc12]

To a stirred suspension of 1-isopropyl-1H-pyrazolo[4,3-c]pyridine-3-carboxylic acid (Preparation 4, 351 mg, 1.71 mmol) in DCM (15 mL) was added N-methoxy-N-methylamine hydrochloride (184 mg, 1.88 mmol) followed by HBTU (713 mg, 1.88 mmol) and triethylamine (0.953 mL, 6.84 mmol) and the reaction stirred at room temperature for 18 hours. Water (3 mL) was added and the reaction stirred vigorously for 10 minutes before separation of the layers through a phase separation cartridge. The organic layer was collected and concentrated in vacuo. The residue was purified using silica gel column chromatography eluting with 95:5:0.5 DCM:MeOH:NH₃ followed by a second chromatography eluting with the same eluant to furnish the title compound (270 mg, 64%).

¹H NMR (400 MHz, CDCl₃): δ ppm 1.63 (s, 6H), 3.56 (br s, 3H), 3.93 (s, 3H), 4.90 (m, 1H), 7.36 (d, 1H), 8.46 (d, 1H), 9.54 (d, 1H).

MS m/z 249 [M+H]⁺

### Preparation 4

### 1-isopropyl-1H-pyrazolo[4,3-c]pyridine-3-carboxylic acid

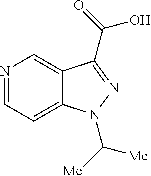 [SMILES: CC(C)n1nc(C(=O)O)c2cnccc21]

To a solution of ethyl 1-isopropyl-1H-pyrazolo[4,3-c]pyridine-3-carboxylate (Preparation 5, 569 mg, 2.44 mmol) in THF (10 mL) was added 1N aqueous NaOH (0.244 mL) and the reaction stirred at room temperature for 18 hours. Further 1N NaOH was added (2.2 mL) and the reaction heated to reflux for 24 hours. The reaction was cooled and quenched with 4M HCl in dioxane (0.6 mL) to pH=3. The organic solvent was removed in vacuo and the aqueous residue was extracted with DCM (25 mL). The organic layer was collected, dried over MgSO₄ and concentrated in vacuo to afford the title compound (351 mg, 70%).

¹H NMR (400 MHz, DMSO-d₆): δ ppm 1.52 (s, 6H), 5.06 (m, 1H), 7.78 (d, 1H), 8.39 (d, 1H), 9.36 (s, 1H).

MS m/z 206 [M+H]⁺

### Preparation 5

### Ethyl 1-isopropyl-1H-pyrazolo[4,3-c]pyridine-3-carboxylate

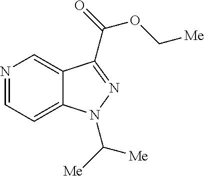 [SMILES: CCOC(=O)c1nn(C(C)C)c2ccncc12]

To a solution of ethyl 1-isopropyl-4,5,6,7-tetrahydro-1H-pyrazolo[4,3-c]pyridine-3-carboxylate (Preparation 6, 802 mg, 3.38 mmol) in 4-isopropylbenzene (15 mL) was added 10% Pd/C (400 mg, 0.38 mmol) and the reaction heated to reflux for 18 hours. The reaction was cooled, filtered and concentrated in vacuo to afford the title compound (569 mg, 72%).

¹H NMR (400 MHz, CDCl₃): δ ppm 1.47 (t, 3H), 1.65 (s, 6H), 4.55 (q, 2H), 4.97 (m, 1H), 7.59 (d, 1H), 8.56 (d, 1H), 9.54 (s, 1H).

MS m/z 234 [M+H]⁺

### Preparation 6

### Ethyl 1-isopropyl-4,5,6,7-tetrahydro-1H-pyrazolo[4,3-c]pyridine-3-carboxylate

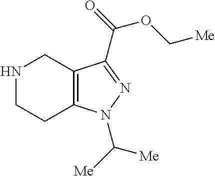 [SMILES: CCOC(=O)c1nn(C(C)C)c2c1CNCC2]

To a stirred solution of 5-tert-butyl 3-ethyl 1-isopropyl-1,4,6,7-tetrahydro-5H-pyrazolo[4,3-c]pyridine-3,5-dicarboxylate (Preparation 7, 6.55 g, 19.41 mmol) in dioxane (20 mL) was added 4M HCl in dioxane (30 mL) and the reaction stirred at room temperature for 1 hour before concentrating in vacuo. The residue was partitioned between saturated aqueous NaHCO₃ solution and EtOAc, the organic layer was collected and the aqueous backwashed with further EtOAc. The organic layers were combined, dried over MgSO₄ and concentrated in vacuo to afford the title compound that solidified on standing (4.6 g, 100%).

¹H NMR (400 MHz, CDCl₃): δ ppm 1.36 (t, 3H), 1.50 (s, 6H), 2.62 (br s, 1H), 2.71 (t, 2H), 3.16 (t, 2H), 4.07 (s, 2H), 4.35 (q, 2H), 4.43 (m, 1H).

MS m/z 238 [M+H]⁺

### Preparation 7

### 5-tert-butyl 3-ethyl 1-isopropyl-1,4,6,7-tetrahydro-5H-pyrazolo[4,3-c]pyridine-3,5-dicarboxylate and 5-tert-butyl 3-ethyl 2-isopropyl-2,4,6,7-tetrahydro-5H-pyrazolo[4,3-c]pyridine-3,5-dicarboxylate

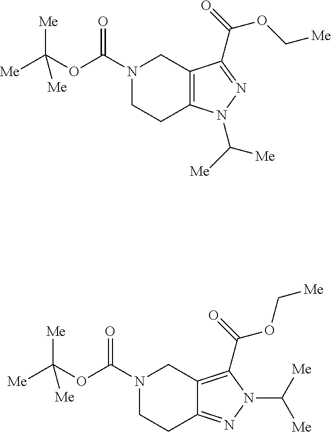 [SMILES: CCOC(=O)c1nn(C(C)C)c2c1CN(C(=O)OC(C)(C)C)CC2 ; CCOC(=O)c1c2c(nn1C(C)C)CCN(C(=O)OC(C)(C)C)C2]

To a stirred solution of 5-tert-butyl 3-ethyl 1,4,6,7-tetrahydro-5H-pyrazolo[4,3-c]pyridine-3,5-dicarboxylate (Preparation 8, 15.6 g, 52.82 mmol) in acetone (150 mL) was added potassium carbonate (19.7 g, 143 mmol) followed by isopropyliodide (7.92 mL, 79.2 mmol). The reaction was stirred at room temperature for 2 hours, further isopropyliodide (7.92 mL, 79.2 mmol) was added and the mixture heated to reflux for 18 hours. The reaction was cooled and the resulting precipitate filtered and the solid collected, washing with acetone. The filtrate was concentrated in vacuo and the residue was purified using silica gel column chromatography eluting with 10-40% EtOAc in pentane to afford the two title compounds in a ratio 1:1.2.

First eluting isomer: 5-tert-butyl 3-ethyl 2-isopropyl-2,4,6,7-tetrahydro-5H-pyrazolo[4,3-c]pyridine-3,5-dicarboxylate (5.43 g, 30%).

¹H NMR (400 MHz, CDCl₃): δ ppm 1.36 (t, 3H), 1.40-1.47 (m, 15H), 2.74 (br s, 2H), 3.66 (br s, 2H), 4.32 (q, 2H), 4.59 (br s, 2H), 5.48 (m, 1H).

Irradiation of the methine proton in ⁱPr group generates an nOe to the two ⁱPr methyl signals only. Molecular mechanics minimisation shows that this is consistent with alkylation on N-2.

Second eluting isomer: 5-tert-butyl 3-ethyl 1-isopropyl-1,4,6,7-tetrahydro-5H-pyrazolo[4,3-c]pyridine-3,5-dicarboxylate (6.55 g, 37%).

¹H NMR (400 MHz, CDCl₃): δ ppm 1.37 (t, 3H), 1.42-1.50 (m, 15H), 2.69 (br t, 2H), 3.70 (br t, 2H), 4.35 (q, 2H), 4.41 (m, 1H), 4.57 (br s, 2H).

Irradiation of the CH₂ at 2.69 ppm generates an nOe to the isopropyl methine at 4.41 ppm.

Molecular mechanics minimisation shows that this is consistent with alkylation on N-1.

### Preparation 8

### 5-tert-butyl 3-ethyl 1,4,6,7-tetrahydro-5H-pyrazolo[4,3-c]pyridine-3,5-dicarboxylate

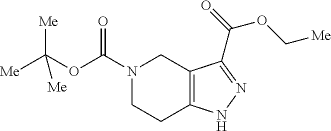 [SMILES: CCOC(=O)c1n[nH]c2c1CN(C(=O)OC(C)(C)C)CC2]

To a stirred solution of tert-butyl (3Z)-3-(2-ethoxy-1-hydroxy-2-oxoethylidene)-4-oxopiperidine-1-carboxylate (Preparation 9, 26 g, 86.86 mmol) in acetic acid (70 mL) was added hydrazine hydrate (4.21 mL, 86.90 mmol) and the reaction was stirred at room temperature for 30 minutes followed by reflux for 1.5 hours. The reaction was cooled and concentrated in vacuo. EtOAc (200 mL) was added to the residue causing a precipitate. The precipitate was filtered and the filtrate washed with saturated aqueous NaHCO₃ solution, dried over MgSO₄ and concentrated in vacuo to afford the title compound containing 1.2 equivalents of AcOH. The material was used directly in the next reaction.

¹H NMR (400 MHz, CDCl₃): δ ppm 1.17 (t, 3H), 1.47 (s, 9H), 2.77 (br t, 2H), 3.70 (br s, 2H), 4.36 (q, 2H), 4.62 (br s, 2H).

MS m/z 591 [2M+H]⁺

### Preparation 9

### tert-butyl (3Z)-3-(2-ethoxy-1-hydroxy-2-oxoethylidene)-4-oxopiperidine-1-carboxylate

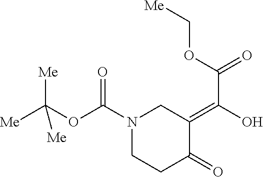 [SMILES: CCOC(=O)/C(O)=C1\CN(C(=O)OC(C)(C)C)CCC1=O]

A solution of tert-butyl 4-oxopiperidine-1-carboxylate (19.9 g, 100 mmol) in Et₂O (100 mL) was added to a solution of LiHMDS (1M in THF, 100 mL, 100 mmol) in Et₂O (100 mL) at −78° C. causing an exotherm to −55° C. After stirring at this temperature for 30 minutes, diethyloxalate (13.6 mL, 100 mmol) was added as a solution in Et₂O (40 mL) and the reaction was allowed to warm to room temperature stirring for 18 hours. The reaction was quenched by the addition of water (50 mL) and the resulting layers were separated. The aqueous layer was neutralised by the addition of 2M HCl and extracted three times with EtOAc (3×100 mL). The combined organic extracts were washed with brine, dried over MgSO₄ and concentrated in vacuo to afford the title compound (26 g, 87%).

¹H NMR (400 MHz, CDCl₃): δ ppm 1.40 (t, 3H), 1.48 (s, 9H), 2.59 (t, 2H), 3.65 (2H, t), 4.37 (2H, q), 4.46 (s, 2H).

### Preparation 10

### (2-aminopyridin-4-yl)(3-isopropylimidazo[1,5-a]pyrazin-1-yl)methanone

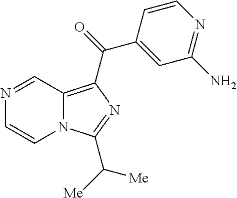 [SMILES: CC(C)c1nc(C(=O)c2ccnc(N)c2)c2cnccn12]

To a solution of {2-[(diphenylmethylene)amino]pyridin-4-yl}(3-isopropylimidazo[1,5-a]pyrazin-1-yl)methanone (Preparation 11, 140 mg, 0.31 mmol) in THF (2 mL) was added 1N citric acid (4 mL) and the reaction was stirred at room temperature for 4 hours. The reaction was concentrated in vacuo and diluted with saturated aqueous NaHCO₃ solution. The aqueous mixture was extracted with EtOAc, the organic layer collected, washed with water, brine, dried over Na₂SO₄ and concentrated in vacuo. The residue was purified using silica gel column chromatography eluting with 1-2% MeOH in DCM to afford the title compound (80 mg, 92%).

LCMS Rt=2.39 minutes MS m/z 282 [M+H]⁺

### Preparation 11

### {2-[(diphenylmethylene)amino]pyridin-4-yl}(3-isopropylimidazo[1,5-a]pyrazin-1-yl)methanone

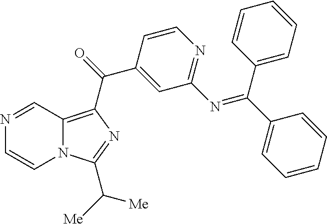 [SMILES: CC(C)c1nc(C(=O)c2ccnc(N=C(c3ccccc3)c3ccccc3)c2)c2cnccn12]

To a solution of 1-iodo-3-isopropylimidazo[1,5-a]pyrazine (Preparation 12, 250 mg, 0.871 mmol) and 2-[(diphenylmethylene)amino]-N-methoxy-N-methylisonicotinamide (Preparation 15, 270 mg, 0.78 mmol) in anhydrous toluene (2.5 mL) cooled to −78° C. was added nBuLi (2.3M, 0.4 mL, 0.91 mmol) and the reaction stirred at this temperature for 30 minutes before being quenched by the addition of saturated aqueous ammonium chloride solution. The organic layer was separated and diluted with EtOAc, washed with water, brine, dried over Na₂SO₄ and concentrated in vacuo. The residue was purified using silica gel column chromatography eluting with 50-60% EtOAc in hexane to afford the title compound (140 mg, 40%).

LCMS Rt=3.45 minutes MS m/z 446 [M+H]⁺

### Preparation 12

### 1-iodo-3-isopropylimidazo[1,5-a]pyrazine

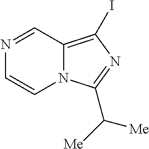 [SMILES: CC(C)c1nc(I)c2cnccn12]

To a solution of 3-isopropylimidazo[1,5-a]pyrazine (Preparation 13, 1.4 g, 8.69 mmol) in anhydrous DMF (12 mL) was added NIS (2 g, 9.13 mmol) and the reaction heated to 60° C. for 3 hours. The reaction was concentrated in vacuo and the residue partitioned between EtOAc and water. The organic layer was collected, washed with Na₂S₂O₃ solution, water, brine, dried over Na₂SO₄ and concentrated in vacuo. The residue was purified using silica gel column chromatography eluting with 10-15% EtOAc in hexane to afford the title compound (1.9 g, 79%).

¹H NMR (400 MHz, DMSO-d₆): δ ppm 1.29 (s, 6H), 3.47-3.54 (m, 1H), 7.55 (d, 1H), 8.24 (d, 1H), 8.71 (s, 1H).

LCMS Rt=2.61 minutes MS m/z 288 [M+H]⁺

### Preparation 13

### 3-isopropylimidazo[1,5-a]pyrazine

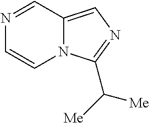 [SMILES: CC(C)c1ncc2cnccn12]

To a solution of 2-methyl-N-(pyrazin-2-ylmethyl)propanamide (Preparation 14, 7 g, 39.1 mmol) in POCl₃ (100 mL) was added DMF (0.1 mL) and the reaction heated at 55° C. for 2 hours. The reaction was cooled and concentrated in vacuo. The residue was quenched by the addition of aqueous ammonia and extracted into DCM. The organic layer was collected, dried over Na₂SO₄ and concentrated in vacuo. The residue was purified using silica gel column chromatography eluting with 1% MeOH in DCM to afford the title compound (1.4 g, 22%).

¹H NMR (400 MHz, DMSO-d₆): δ ppm 1.32 (s, 6H), 3.46-3.53 (m, 1H), 7.48 (d, 1H), 7.72 (s, 1H), 8.19 (d, 1H), 8.98 (s, 1H).

LCMS Rt=1.97 minutes MS m/z 162 [M+H]⁺

### Preparation 14

### 2-methyl-N-(pyrazin-2-ylmethyl)propanamide

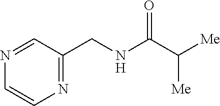 [SMILES: CC(C)C(=O)NCc1cnccn1]

To a solution of 1-pyrazin-2-ylmethanamine hydrochloride (15 g, 103.4 mmol) in MeOH (15 mL) was added KOH (5.8 g, 103.4 mmol) at room temperature before cooling to 0° C. Isobutyric anhydride (25.7 mL, 155.16 mmol) was added dropwise over 10 minutes. Further KOH was added (5.8 g, 103.4 mmol) followed by isobutyric anhydride (25.7 mL, 155.16 mmol) and the reaction slowly warmed to room temperature for 18 hours. The reaction was reduced to low volume (30 mL) in vacuo and extracted with DCM. The organic layer was collected, dried over Na₂SO₄ and concentrated in vacuo. The residue was purified using silica gel column chromatography eluting with 3-5% MeOH in DCM to afford the title compound (7 g, 49%).

¹H NMR (400 MHz, DMSO-d₆): δ ppm 1.04 (s, 6H), 2.44 (d, 1H), 4.38 (m, 1H), 8.41 (br s, 1H), 8.52-8.62 (m, 3H).

LCMS Rt=1.35 minutes MS m/z 180 [M+H]⁺

### Preparation 15

### 2-[(Diphenylmethylene)amino]-N-methoxy-N-methylisonicotinamide

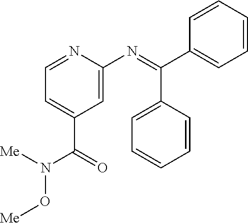 [SMILES: CON(C)C(=O)c1ccnc(N=C(c2ccccc2)c2ccccc2)c1]

Benzophenone imine (2.17 g, 12.0 mmol) was added to 2-bromo-N-methoxy-N-methylisonicotinamide (2.45 g, 10.0 mmol), tris(dibenzylideneacetone)dipalladium (458 mg, 0.50 mmol), 2-di-tert-butylphosphino-2′,4′,6′-triisopropylbiphenyl (552 mg, 1.30 mmol) and sodium t-butoxide (2.40 g, 25.0 mmol) in toluene (40 mL). The mixture was stirred at room temperature for 2 hours. The reaction mixture was diluted with DCM and filtered through Arbocel™. The filtrate was washed with water (100 mL), the organic phase was dried over sodium sulphate and evaporated in vacuo. The crude material was purified by silica gel column chromatography eluting with a gradient of heptanes:EtOAc 100:0 to 30:70 to afford the title compound as an orange gum (2.44 g, 71%).

¹H NMR (400 MHz, DMSO-d₆): δ ppm 3.14 (br s, 3H), 3.30 (br s, 3H), 6.76 (m, 1H), 7.02 (dd, 1H), 7.11-7.19 (m, 2H), 7.27-7.36 (m, 3H), 7.46-7.54 (m, 2H), 7.59 (m, 1H), 7.66-7.73 (m, 2H), 8.32 (dd, 1H).

### Preparation 16

### Ethyl (2-cyclopropyl-1,3-oxazol-4-yl)acetate

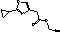 [SMILES: CCOC(=O)Cc1coc(C2CC2)n1]

Ethyl 4-chloroacetoacetate (20.0 g, 122.0 mmol) was added to cyclopropanecarboxamide (3.52 g, 41.5 mmol) in toluene (100 mL) and 1,4-dioxane (100 mL). The mixture was refluxed at 120° C. for 17 hours then evaporated in vacuo. The crude solid was purified by silica gel column chromatography eluting with 80:20 petroleum ether: EtOAc to afford the title compound as a white solid (50%, 4.00 g).

¹H NMR (300 MHz, DMSO-d₆): δ ppm 0.80-1.00 (m, 4H), 1.20 (t, 3H), 2.10 (m, 1H), 3.50 (s, 2H), 4.10 (q, 2H), 7.80 (s, 1H).

### Preparation 17

### (2-Cyclopropyl-1,3-oxazol-4-yl)acetic acid

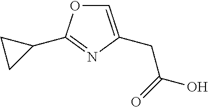 [SMILES: O=C(O)Cc1coc(C2CC2)n1]

Lithium hydroxide monohydrate (7.83 g, 186.7 mmol) was added to ethyl (2-cyclopropyl-1,3-oxazol-4-yl)acetate (Preparation 16, 7.00 g, 35.9 mmol) in THF (200 mL) and water (100 mL). The mixture was stirred at room temperature for 2 hours then the reaction mixture volume was reduced to one third by evaporation in vacuo. The aqueous residue was acidified using aqueous 1M HCl then extracted with EtOAc (200 mL). The organic phase was evaporated in vacuo and the crude material was triturated with diethyl ether (100 mL) to afford the title compound as a white solid (66%, 4.00 g).

¹H NMR (300 MHz, CDCl₃): δ ppm 1.05 (m, 4H), 2.10 (m, 1H), 3.60 (s, 2H), 7.40 (s, 1H), 10.00 (br s, 1H).

### Preparation 18

### tert-Butyl[4-(trifluoromethyl)-1H-pyrazol-1-yl]acetate

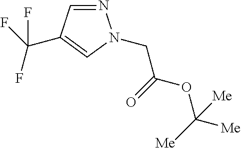 [SMILES: CC(C)(C)OC(=O)Cn1cc(C(F)(F)F)cn1]

Potassium carbonate (7.67 g, 55.56 mmol) was added to 4-(trifluoromethyl)-1H-pyrazole (2.518 g, 18.52 mmol) in dry DMF (20 mL) at 25° C. and the mixture was stirred for 20 minutes. Ethyl bromoacetate (2.06 mL, 18.52 mmol) was added then the mixture was stirred for 2 days at room temperature. The reaction mixture was neutralized with aqueous HCl (1.0 M), extracted with ether (40 mL) and the organic extract was washed with brine (30 mL), dried over sodium sulfate then evaporated in vacuo. The residue was purified by silica gel column chromatography eluting with hexane:EtOAc 90:10 to afford the title compound as a yellow solid (24%, 1.32 g).

LCMS Rt=3.64 minutes MS m/z 251 [M+H]⁺

### Preparation 19

### [4-(Trifluoromethyl)-1H-pyrazol-1-yl]acetic acid

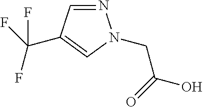 [SMILES: O=C(O)Cn1cc(C(F)(F)F)cn1]

Trifluoroacetic acid (10 mL) was added to tert-butyl [4-(trifluoromethyl)-1H-pyrazol-1-yl]acetate (Preparation 18, 1.3 g, 5.2 mmol) in dry DCM (10 mL) and the mixture was stirred for 18 hours at 25° C. Then the mixture was evaporated in vacuo and the residue was purified by trituration with diethyl ether:pentane (1:9, 2 mL) to afford the title compound as a white solid (79%, 800 mg).

LCMS Rt=1.39 minutes MS m/z 193 [M−H]⁻

### Biological Activity

Isolated TRK Enzyme assays use the HTRF KinEASE-TK kit (Cisbio Cat#62TK0PEJ) with recombinant His-tagged cytoplasmic domains of each TRK receptor sourced from Invitrogen (see table below). This activity-assay measures the phosphorylation of tyrosine residues within a substrate from the HTRF kit which has been validated by Cisbio for a variety of tyrosine kinases including the TRK receptors.

### Assay Details:

| | Invitrogen | Amino | FAC | FAC | Assay Reaction |
|---|---|---|---|---|---|
| Target | Cat# | acids | enzyme | ATP | Time |
| TRKA | PV3144 | aa 441- | 4 nM | 40 uM | 35 min |
| | (NTRK1) | 796 | | | |
| TRKB | PV3616 | aa 526- | 1 nM | 1.4 uM | 40 min |
| | (NTRK2) | 838 | | | |
| TRKC | PV3617 | aa 510- | 10 nM | 15 uM | 30 min |
| | (NTRK3) | 825 | | | |

0.5 mM stock solutions of test compounds are prepared and serially diluted in 100% DMSO. A standard curve using the compound of Example 135 disclosed in WO2005/116035 of 150 uM is also prepared on each test plate. High percentage effect (HPE) is defined by 150 uM (using the compound of Example 135 as disclosed in WO2005/116035) and 0% effect (ZPE) is defined by 100% DMSO. Greiner low volume black plates containing 0.2 ul of serially diluted compound, standard and HPE/ZPE are created using the Bravo nanolitre dispenser.

1× enzyme buffer is prepared from 5× Enzymatic Buffer from the Cisbio KinEASE TK kit using MilliQ water. The buffer is then supplemented with 10 mM MgCl and 2 mM DTT (both from Sigma). In the case of TRKB, the buffer is also supplemented with 125 nM Supplement Enzymatic Buffer (SEB) from the Cisbio kit.

2×FAC of enzyme and 2×FAC ATP diluted in 1× complete enzyme buffer is incubated at room temperature for 20 minutes to preactivate the enzyme. Following this preactivation step, 5 ul/well of enzyme+ATP mix is added using a Multidrop Micro to the assay plate, spotted with 0.2 ul 100% DMSO compound. This is left for 20 mins at room temperature before adding 5 ul of 2 uM TK-substrate-Biotin (from the Cisbio kit) diluted in 1× enzyme buffer (1 uM FAC) using the Multidrop Micro. The reaction is incubated at room temperature for the optimized assay reaction time (see table). The reaction is stopped by adding 10 ul/well HTRF Detection Buffer containing 0.25 uM Streptavidin-XL665 (0.125 uM FAC) and 1:200 TK Antibody-Cryptate using a Multidrop.

After the Detection Reagent addition, plates are covered and incubated at room temperature for 60 minutes. HTRF signal is read using an Envision reader, measured as a ratio of emissions at two different wavelengths, 620 nm and 665 nm. Any compound that inhibits the action of the TRK kinase will have a lower fluorescence ratio value 665/620 nM than compounds which do not inhibit the TRK kinase. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

Cell Based Assays were carried out using Cell lines from DiscoveRx utilising their PathHunter technology and reagents in an antagonist assay:

| | DiscoveRx cell | Cognate |
|---|---|---|
| Target | line Cat# | Neurotrophin |
| TRKA | 93-0462C3 | NGF |
| TRKA co expressed | 93-0529C3 | NGF |
| with p75 | | |
| TRKB | 93-0463C3 | BDNF |
| TRKB co expressed | 93-0530C3 | BDNF |
| with p75 | | |
| TRKC | 93-0464C3 | NT3 |
| TRKC co expressed | 93-0531C3 | NT3 |
| with p75 | | |

The assays are based upon DiscoveRx's proprietary Enzyme Fragment Complementation (EFC) technology. In the case of the TRK cell lines, the enzyme acceptor (EA) protein is fused to a SH2 protein and the TRK receptor of interest has been tagged with a Prolink tag.

Upon neurotrophin binding, the TRK receptor becomes phosphorylated, and the tagged SH2 protein binds. This results in functional complementation and restored β-Galactosidase activity which is can be measured using the luminescent Galacton Star substrate within the PathHunter reagent kits.

Generally, small molecule inhibitors bind to the kinase domain so are not competing with the neurotrophin (agonist) which binds to an extracellular site. This means that the IC₅₀ is a good measure of affinity and should be unaffected by concentration neurotrophin stimulant.

Cryopreserved PathHunter cells are used from either in-house produced batches or bulk batches bought directly from DiscoveRx. Cryopreserved cells are resuscitated, spun 1000 rpm for 4 min to remove freezing media, and resuspended in MEM+0.5% horse serum (both Invitrogen) to 5e⁵ cells/ml. The cells are then plated using a Multidrop into Greiner white tissue culture treated plates at 20 ul/well and incubated for 24 h at 37° C., 5% CO₂, high humidity. On the day of the assay, the cell plates are allowed to cool to room temperature for 30 min prior to the assay.

4 mM stock solutions of test compounds are prepared and serially diluted in 100% DMSO. A standard curve using the compound of Example 135, WO2005/116035 at a top concentration of 150 uM is also prepared on each test plate. High percentage effect (HPE) is defined by 150 uM of the compound of Example 135, WO2005/116035 and 0% effect (ZPE) is defined by 100% DMSO. Plates containing 1 ul of serially diluted compound, standard and HPE/ZPE are diluted 1/66 in assay buffer (PBS minus Ca²⁺, minus Mg²⁺ with 0.05% pluronic F127) using a Wellmate. Using a Platemate Plus, 5 ul of 1/66 diluted test compounds is then transferred to the cell plate and allowed to reach equilibrium by incubating for 30 min at room temperature before addition of agonist stimulus: 10 ul/well of 2 nM (0.571 nM FAC) of the cognate neurotrophin (Peprotech) diluted in agonist buffer (HBSS with 0.25% BSA). Final assay concentration of the test compounds is 8.66 μM, (the compound of Example 135, WO2005/116035 FAC is 0.325 uM). The plates are left at room temperature for a further 2 hours before addition of 10 ul of the DiscoveRx PathHunter detection reagent (made up by adding 1 part Galacton Star, 5 parts Emerald II and 19 parts Cell Assay Buffer as per the manufacturer's instructions).

After reagent addition, plates are covered and incubated at room temperature for 60 minutes. Luminescence signal is read using an Envision. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

### Brain Penetration Assays

### In Vitro

MDCK-BCRP: MDCK-BCRP data may be collected according to the method described in “A 96-Well Efflux Assay To Identify ABCG2 Substrates Using a Stably Transfected MDCK II Cell Line” http://pubs.acs.orq/doi/full/10.1021/mp050088t Yongling Xiao, Ralph Davidson, Arthur Smith, Dennis Pereira, Sabrina Zhao, John Soglia, David Gebhard, Sonia de Morais, and David B. Duignan, Mol. Pharm., 2006, 3 (1), pp 45-54.

MDCK-MDR1: MDCK-MDR1 data may be collected according to the method described in “Are MDCK Cells Transfected with the Human MDR1 Gene a Good Model of the Human Intestinal Mucosa?” http://www.springerlink.com/content/qfhqlqbr4fnp3khf/fulltext.pdf

Fuxing Tang, Kazutoshi Horie, and Ronald T. Borchardt, Pharmaceutical Research, Vol. 19, No. 6, June 2002.

### In Vivo

Brain penetration may be measured according to the method described in “Assessing brain free fraction in early drug discovery”. Read, K; Braggio, S., Expert Opinion Drug Metab Toxicol. (2010) 6 (3) 337-344.

Below are TrkA IC₅₀ data generated using the PV3144 TrkA enzyme assay. Where more than one reading was taken, the arithmetic mean is presented.

| Example | TrkA IC₅₀ (nM) |
|---|---|
| 1 | 21 |
| 2 | 20.4 |
| 3 | 16.6 |

All publications cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention.

Accordingly, the invention is limited only by the following claims.

1. A compound of Formula IA or IB: 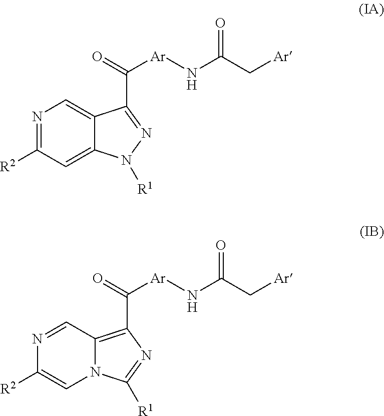
or a pharmaceutically acceptable salt thereof, wherein
R¹ is C₂₋₄ alkyl optionally substituted by 1 or 2 OH, optionally wherein a methylene group is replaced by an oxetane group,
or R¹ is C₄₋₆ cycloalkyl optionally substituted by OH;
R² is H or NH₂;
Ar is a ring system selected from 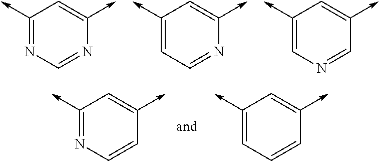
which ring system is optionally substituted on a carbon atom by CN, C₁₋₃ alkyl, C₁₋₃ alkoxy, or C₃₋₆ cycloalkyloxy;
and Ar′ is a ring system selected from 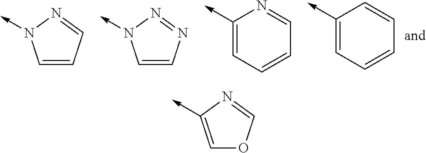
which ring system is optionally substituted on a carbon atom by 1 or 2 substituents independently selected from:
halo, ═O, CN, C₁₋₃ alkyl optionally substituted by one or more F, C₁₋₃ alkoxy optionally substituted by one or more F, C₃₋₆ cycloalkyl, C₃₋₆ cycloalkyloxy and SO₂(C₁₋₃ alkyl).

2. The compound according to claim 1 or a pharmaceutically acceptable salt thereof wherein R¹ is selected from:

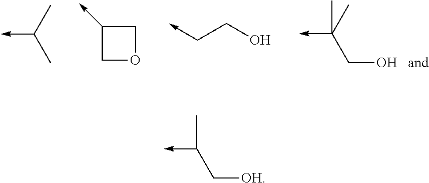

3. The compound according to claim 1 or a pharmaceutically acceptable salt thereof wherein Ar′ is a ring system which ring is selected from: 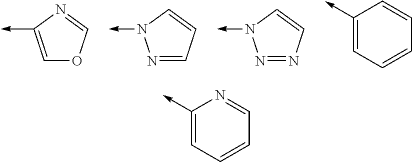
and which ring is optionally substituted by 1 or 2 substituents independently selected from F, Cl, ═O, CN, CF₃, OCF₃, CH₃, isopropyl, OCH₃, cyclopropyl, and

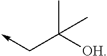

4. The compound according to claim 1 or a pharmaceutically acceptable salt thereof wherein Ar is

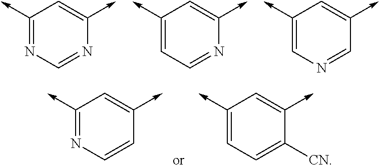

5. The compound according to claim 1 or a pharmaceutically acceptable salt thereof 4 wherein R¹ is 1-hydroxy-2-methylpropan-2-yl, 1-hydroxypropan-2-yl or isopropyl.

6. The compound according to claim 1 or a pharmaceutically acceptable salt thereof wherein R² is H.

7. A compound selected from:
2-(2-cyclopropyl-1,3-oxazol-4-yl)-N-{4-[(1-isopropyl-1H-pyrazolo[4,3-c]pyridin-3-yl)carbonyl]pyridin-2-yl}acetamide;
2-(4-cyanophenyl)-N-{4-[(1-isopropyl-1H-pyrazolo[4,3-c]pyridin-3-yl)carbonyl]pyridin-2-yl}acetamide; and
N-{4-[(3-isopropylimidazo[1,5-a]pyrazin-1-yl)carbonyl]pyridin-2-yl}-2-[3-(trifluoromethyl)-1H-pyrazol-1-yl]acetamide;
or a pharmaceutically acceptable salt thereof.

8. A pharmaceutical composition comprising a compound of the formula (IA or IB) or a pharmaceutically acceptable salt thereof, as defined in claim 1, and a pharmaceutically acceptable carrier.

9.-13. (canceled)

14. A method of treatment of a mammal, to treat a disease for which an Trk receptor antagonist is indicated, comprising treating said mammal with an effective amount of a compound of the formula (IA or IB) or a pharmaceutically acceptable salt thereof, as defined in claim 1.

15. A method of treatment of pain or cancer in a mammal, comprising treating said mammal with an effective amount of a compound of the formula (IA or IB) or a pharmaceutically acceptable salt thereof, as defined in claim 1.

16. A method of treatment of a mammal, to treat a disease for which an Trk receptor antagonist is indicated, comprising treating said mammal with an effective amount of a compound according to claim 1 in combination with a further drug substance.
